(19) United States
(12) Patent Application Publication
(10) Pub. No.: US 2005/0252781 A1
(43) Pub. Date: Nov. 17, 2005

(54) Method for packing electrochemically-deposited elements

(76) Inventors: Yuval Golan, Omer (IL); Brenda Laster, Meitar (IL); Gad Shani, Omer (IL); Moshe Faraggi, Omer (IL); Roberto Tulman, Or-Yehuda (IL)

Correspondence Address: NIXON & VANDERHYE, PC, 901 NORTH GLEBE ROAD, 11TH FLOOR, ARLINGTON, VA, 22203, US

(21) Appl. No.: 10/499,155

(22) PCT Filed: Dec. 17, 2002
(86) PCT No.: PCT/IL02/01013

§ 371 Date: May 26, 2005

(30) Foreign Application Priority Data
Dec. 20, 2001 (IL) 147199

(51) Int. Cl. C25D 5/02

(52) U.S. Cl. 205/131

(57) ABSTRACT

A method for densely packaging an element inside a casing, such that the element-containing casing is suitable for use in the preparation of a radiation source, comprising providing a solution containing ions of said element, positioning a working electrode and at least one counter electrode in contact with said solution, connecting said working electrode and said at least one counter electrode to the negative and positive poles of a power source, respectively, passing an electrical current between said electrodes to electrochemically deposit said element on said working electrode in a geometrical form corresponding to the form of the interior of the casing and concurrently or sequentially loading said casing with said element.

### FIELD OF THE INVENTION

The present invention relates to a method for packaging electrochemically-deposited elements inside casings that are intended for use as radiation sources. Particularly, the present invention provides a method for packing elemental samarium inside casings, and to samarium-containing casings obtained thereby, which are useful as brachytherapy seeds.

### BACKGROUND OF THE INVENTION

There exists a need, in many different technological areas (for example, medical engineering), for densely packaging metals that can be converted into radioactive isotopes, inside suitable casings intended for use as radiation sources. In general, it is preferred to load the casing with the elemental form of the metal, rather than with salts or oxide thereof, since the efficacy of the radiation source depends on the concentration of the radioactive isotope contained therein.

For example, such a need exists in relation to brachytherapy. Brachytherapy is a method of radiation therapy involving the implantation of a small radiation source containing a radioactive isotope within a particular body region. The combination of features required for a radioactive isotope to function as a useful brachytherapy radiation source is not easily met. Specifically, the radioisotope must have a long half-life and an appropriate decay profile, and, in addition, it must be easily and effectively encapsulated within available casings, to form the “brachytherapy seed” (this term is used in the art to define the small canisters, generally made of titanium, that contain the radioactive isotope). The commercially available brachytherapy seeds contain radioactive isotopes such as iodine-125, palladium-103 and iridium-192, having half-lives of 60 days, 17 days and 74 days, respectively.

US Statutory Invention Registration no. H669 discloses the production of a samarium-145 radiation source useful for brachytherapy applications, by neutron irradiation of samarium-144. Samarium-145 is characterized by several valuable properties, including a relatively long half-life (340 days). The publication also describes titanium tubes that were loaded with samarium oxide, Sm₂O₃, the commercially available form of enriched samarium, which, following activation by means of neutron irradiation, may be suitable for use as brachytherapy seeds. The publication does not disclose a method for effectively and densely packing elemental samarium, in place of samarium oxide, in said titanium tubes.

Jundhale et al. [Materials Chemistry and Physics 27, p. 265-278 (1991)] describe the preparation of elemental samarium by means of an electrochemical deposition of samarium films from aqueous solutions.

It is an object of the present invention to provide an efficient method allowing dense packing of different elements inside casings that have different geometrical shapes, such that the element-containing casing is suitable for use in the preparation of a radiation source.

It is yet another object of the invention to provide an efficient method allowing dense packaging of elemental samarium inside canisters suitable for use in brachytherapy applications.

It is yet another object of the present invention to densely pack elemental samarium inside a casing under conditions allowing minimal exposure of the metal to air, in order to maintain the metal in an elemental form and to avoid oxidation thereof.

### SUMMARY OF THE INVENTION

The invention provides a method for densely packaging an element inside a casing, such that the element-containing casing is suitable for use in the preparation of a radiation source, comprising providing a solution containing ions of said element, positioning a working electrode and at least one counter electrode in contact with said solution, connecting said working electrode and said at least one counter electrode to the negative and positive poles of a power source, respectively, passing an electrical current between said electrodes to electrochemically deposit said element on said working electrode in a geometrical form corresponding to the form of the interior of the casing and concurrently or sequentially loading said casing with said element.

Subsequently, before use, the casing may be sealed and activated to convert the element contained therein into a radioactive isotope.

The term “concurrently loading” refers to the case wherein the element is electrochemically deposited inside the casing. The term “sequentially loading” refers to the case wherein the element is electrochemically deposited outside the casing, and is later inserted into the casing.

Any element that may be electro-deposited from a solution, and may be converted into a radioactive isotope, may be packed according to the method of the present invention. Preferably, the element is a metal selected from the group consisting of samarium, thulium, palladium, tellurium, iridium and silver. Most preferably, the element that is packed inside the casing is samarium (¹⁴⁴Sm), and the casing is subsequently sealed and activated by means of a neutron irradiation to produce the radioactive isotope ¹⁴⁵Sm.

According to the present invention, the element-containing casing, following the conversion of the element into a radioactive isotope, is intended for use as a radiation source. The casing is generally made of a material that is transparent to the radiation (e.g., to γ or β radiation), which is expected to be emitted from the radioactive isotope. Preferably, the material of which the casing is made is composed of elements having a relatively low atomic number, said atomic number being preferably in the range of 12 to 28. In addition, the material of which the casing is made should be chemically inert in relation to the environment in which the element-containing casing is intended to function, in order to eliminate the danger of undesired chemical reactions, such as corrosion attack, between the casing and the environment.

According to a preferred embodiment of the invention, the casing is provided in the form of a cylindrically shaped canister, suitable for use in the preparation of an implantable radiation source for brachytherapy applications, and the element to be packed is electrochemically deposited in an essentially cylindrical form corresponding to the interior of said canister. The canister is made of a material, which, in addition to the properties listed above, is also well tolerated by the body. Preferably, the canister is made of titanium.

According to a first preferred embodiment of the invention, the counter electrode positioned in the solution is in the form of a cylindrical surface, within which the working electrode, preferably provided in the form of a wire, is coaxially arranged, whereby, following the passage of an electrical current between said electrodes, the element is electrochemically deposited on said wire, such that a solid body made of said element is obtained, said body having an essentially cylindrical form, wherein the symmetry axis of said body coincides with said wire. Most preferably, the counter electrode is in the form of a cylindrical grid surface, thus allowing an efficient mass transfer of the ions of said element to the working electrode.

According to a second preferred embodiment of the invention, the method for densely packing electrochemically-deposited element inside a casing comprises positioning in the solution a working electrode provided in the form of a perforated plate, wherein each hole of said plate contains a casing made of a conductive material, the length and the cross-section of said casing being essentially the same as the thickness of said plate and cross-section of said hole, respectively, such that said casings are fixedly positioned in said holes, whereby, following the passage of an electrical current between the electrodes, the element is electrochemically deposited inside said casings. Preferably, said casings are provided in the form of tubes that are made of titanium.

According to a particularly preferred variant of said second embodiment, the working electrode is symmetrically positioned in the space between two counter electrodes that are placed parallel to each other. Preferably, said working electrode is caused to oscillate backwards and forwards towards and away from each of said counter electrodes in turn, thus assuring a sufficient concentration of the ions of the element to be packed in the vicinity of the working electrode and particularly, inside the interior of the casings placed therein.

According to a particularly preferred variant of said second embodiment, the passage of the electrical current is performed as follows:
passing an electrical current of magnitude I_{forward} for a period of time t_{forward}, to electrochemically deposit the material in the interior of the casings;
reversing the polarity of the electrodes and passing a reverse current of magnitude Iᵣₑᵥₑᵣₛₑ for a period of time tᵣₑᵥₑᵣₛₑ, wherein I_{forward}<Iᵣₑᵥₑᵣₛₑ and t_{forward}>tᵣₑᵥₑᵣₛₑ, reversing the polarity of the electrodes,
and repeating said steps to obtain a uniform deposit of said material inside the casing.

In another aspect, the present invention provides a radiation source comprising ¹⁴⁵Sm packed inside a casing, wherein said ¹⁴⁵Sm is obtained by neutron-irradiating ¹⁴⁴Sm which was electrochemically deposited from a solution of a samarium salt, and wherein the density of said ¹⁴⁵Sm in said casing is greater than 1.5 g Sm/cm³, and preferably greater than 3 g Sm/cm³, and most preferably greater than 5 g Sm/cm³. Preferably, the casing is a titanium tube suitable for brachytherapy applications.

All the above and other characteristics and advantages of the present invention will be further understood from the following illustrative and non-limitative description of preferred embodiments thereof.

### BRIEF DESCRIPTION OF THE DRAWINGS

FIG. 1 schematically illustrates a samarium cylinder prepared according to one embodiment of the invention.

FIGS. 2a and 2b illustrate the structure of the working electrode according to a second preferred embodiment of the invention. FIG. 2b represents a transverse section of the electrode, taken along line A-A of FIG. 2a.

FIG. 3 shows the arrangement of the electrodes according to the second embodiment of the invention.

### DETAILED DESCRIPTION OF PREFERRED EMBODIMENTS

The invention provides an electroplating method, for densely packing an electrochemically-deposited element inside a casing, such that the element-containing casing is suitable for use in the preparation of a radiation source, comprising providing a solution containing ions of said element, positioning a working electrode and at least one counter electrode in contact with said solution, connecting said working electrode and said at least one counter electrode to the negative and positive poles of a power source, respectively, passing an electrical current between said electrodes to electrochemically deposit said element on said working electrode in a geometrical form corresponding to the form of the interior of the casing, and concurrently or sequentially loading said casing with said element.

In a preferred embodiment of the invention, the electrochemically-deposited material is samarium (¹⁴⁴Sm).

Preferably, the solution used in performing the method of the present invention is an aqueous solution, which is preferably prepared from deionized water. Any water-soluble samarium salt may be used as a source for the samarium ions. Preferably, the samarium salt dissolved in the solution is either samarium oxide, Sm₂O₃, or samarium nitrate, Sm(NO₃)₃, most preferred being Sm₂O₃. The preferred concentration of Sm₂O₃ in the aqueous solution is in the range of 10-50 g/liter, and more preferably in the range of 15-25 g/liter. Samarium nitrate may be used in corresponding molar concentrations. In the case that the casing that is loaded with the elemental samarium according to the invention is intended for use as an implantable radiation source in brachytherapy applications, enriched samarium salt should be used as a source for the samarium ions, in order to allow the subsequent production of the radioactive samarium-145. Enriched samarium is commercially available in the oxide form.

The electrochemical reduction of Sm³⁺ to give elemental samarium is preferably performed under acidic conditions, preferably at a pH in the range of 1.5 to 5, more preferably at a pH in the range of 2 to 3. The pH is preferably adjusted to the desired range by means of an acid, which is preferably nitric acid. Other acids may be used, provided that they do not form a precipitate in the presence of samarium ions.

The electrochemical reduction of Sm⁺³ to give elemental samarium is preferably carried out in the presence of a complex-forming anion, which is a ligand capable of forming a complex with Sm⁺³, such that the deposition potential of samarium is reduced, under acidic conditions, and is preferably shifted to a value in the range of −0.50 to −0.80 V vs. SCE (Standard Calomel Electrode), and more preferably to a value in the range of −0.60 to −0.70 V vs. SCE. Preferably, the complex-forming anion is selected from the group consisting of the ligands tartrate, oxalate, citrate, EDTA and thiocyanate, most preferably the tartrate ligand. The molar ratio between the complex-forming anion present in the solution and the samarium ion is preferably in the range of 1:1 to 5:1.

Preferably, the electrochemical reduction of Sm⁺³ to give elemental samarium is carried out at a temperature in the range of 25 to 60° C., and more preferably in the range of 30 to 40° C.

Preferably, the electrochemical reduction of Sm⁺³ to give elemental samarium is carried out in a solution containing preservatives and other additives such as brighteners and levelers, which are commonly used in electroplating baths.

According to a particularly preferred embodiment of the present invention, the casing to be loaded with elemental samarium is intended, following suitable activation by means of neutron irradiation to convert the samarium-144 into the radioactive isotope samarium-145, to be used as an implantable radiation source in brachytherapy application. Preferably, the casing is provided in the form of a cylindrically shaped canister, and most preferably in the form of a small titanium tube. According to a particularly preferred embodiment of the invention, the canister is provided in the form of a titanium tube, having an internal diameter of about 0.4 to 0.7 mm and a length of few millimeters. Such titanium tubes are commercially available (Uniform Tubes Inc., South Plainfield, N.J. 07080, USA). Following the packing of elemental samarium inside said tube, according to the embodiments described herein below, the tube is sealed, preferably by laser welding or other methods known in the art. Suitable techniques include, for example, laser welding, electron beam welding, crimp welding, gas tungsten arc welding, gas metal arc welding, flux cored arc welding, shielded metal arc welding or submerged arc welding. The activation of the radiation source may be performed in accordance with the description of US Statutory Invention Registration H669, which is incorporated herein by reference. In general, the strength of the source will vary in accordance with its clinical utility. For example, for brain tumors, a 7 to 10 mCi source will be required to accommodate the larger tumor at the time of diagnosis. Activation of 10¹⁹ atoms of ¹⁴⁴Sm to produce ¹⁴⁵Sm will be accomplished by means of irradiation at a neutron flux of 10¹⁵ neutrons/cm²·s, for 15.5 days.

According to a first embodiment of the invention, the method for packing elemental samarium inside a casing involves the electrochemical deposition of said samarium in a geometrical form corresponding to the form of the interior of the casing, and subsequently, loading said casing with said electrochemically deposited elemental samarium.

According to the first embodiment of the invention, the counter electrode positioned in the solution is in the form of a cylindrical grid surface, which is preferably made of a material selected from the group consisting of Pt, platinized Pt or graphite. Preferably, the length and the diameter of said cylindrical surface, which constitutes the counter electrode, are in the ranges of 7 to 13 cm and 2 to 4 cm, respectively.

According to the first embodiment of the invention, the working electrode is provided in the form of a wire, which is coaxially positioned within the cylindrical space defined by the counter electrode. Preferably, said wire is made of graphite, although wires made of metals such as Ti may also be used. The diameter of the wire is preferably in the range of 10 to 50 μm.

The working electrode and the counter electrode positioned in the samarium containing solution are electrically connected to the negative and positive poles of a suitable power source, respectively. Typical current density applied according to the present invention is in the range of 0.5 to 30 mA/cm², in order to avoid hydrogen evolution at the cathode. The cylindrical symmetry of the arrangement of the electrodes according to this embodiment of the invention causes the samarium, which is reduced according to the following cathode reaction:
Sm⁺³+3e→Sm
to coat the wire that functions as the working electrode (cathode), such that a solid body made of samarium is obtained, said body having an essentially cylindrical form, wherein the symmetry axis of said body essentially coincides with said wire.

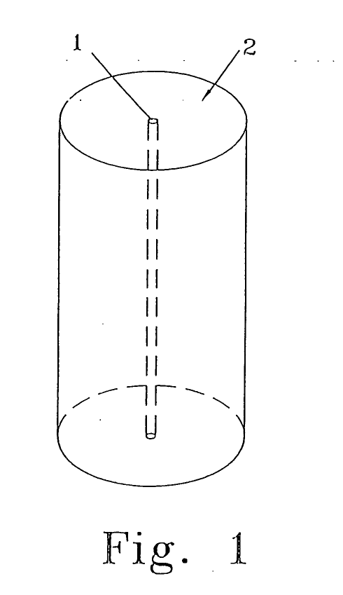

The electrodeposited samarium cylinder produced according to this embodiment is illustrated in FIG. 1, wherein numeral 1 indicates the wire, and numeral 2 indicates the elemental samarium coating. In practice, the growth of the cylindrical samarium coating 2 on the wire 1 is monitored using optical means, in order to assure that the diameter of the samarium cylinder produced is slightly smaller than the diameter of the canister in which said cylinder is to be packed. Preferably, the diameter of said cylindrical body is about 0.38 mm, such that transverse sections of said cylindrical body can be easily and effectively inserted into a 0.4 mm diameter canister intended for use as a brachytherapy seed. The length of the samarium cylinder corresponds to the length of the wire used as the working electrode. Thus, in practice it is preferable to use a relatively long wire, such that following the removal of the samarium cylinder from the solution, and subsequent washing and drying, transverse sections of said samarium cylinder may be prepared, which may be easily and effectively inserted into the canister.

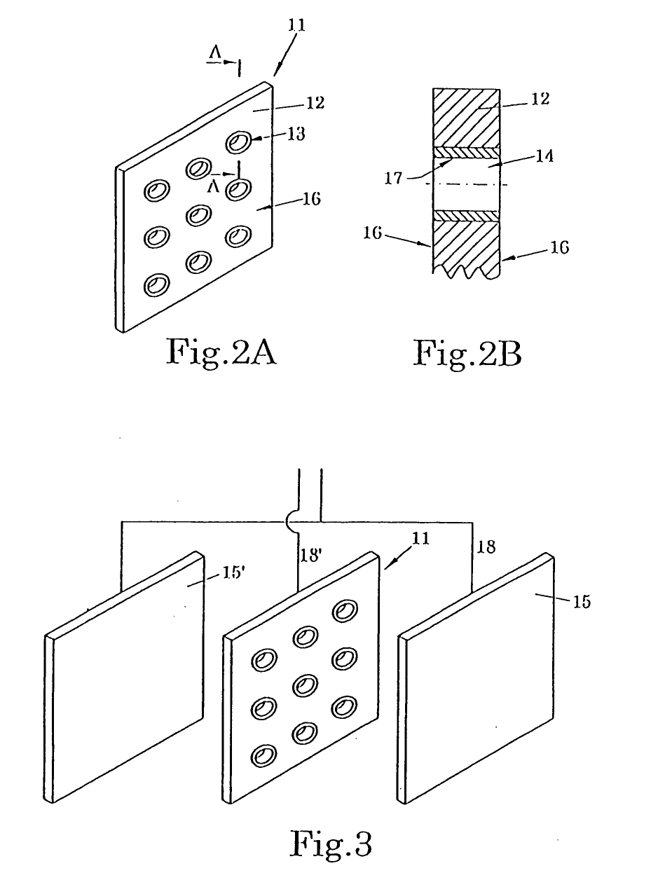

According to a second embodiment of the invention, the method for densely packing elemental samarium inside a casing involves the electrochemical deposition of said samarium in a geometrical form corresponding to the form of the interior of the casing, by causing the electrochemical deposition to occur directly inside said casing. A particularly useful mode for carrying out this second embodiment of the invention is illustrated in FIGS. 2a, 2b and 3.

FIGS. 2a and 2b show the structure of the working electrode to be used according to this embodiment of the invention. The working electrode 11 is provided in the form of a perforated plate 12, wherein each hole 13 of said plate contains a titanium tube 14, as shown in FIG. 2b, the length and the cross-section of said tube being essentially the same as the thickness of said plate and cross-section of said hole, respectively, such that said tubes are fixedly positioned in said holes. The working electrode 11 is made of a soft, ductile conductive material such as copper, gold and silver. Electrodes that are made of plastic materials containing high percentage of metals may also be used as the working electrode, such as conductive epoxy loaded with Ag particles (ca. 80% w/w).

Preferably, the surface 16 of the perforated plate which constitutes the working electrode is electrically insulated by means of appropriate coating, such as teflon, halar or pvc-based materials, or other lacquers, thereby causing the samarium to be deposited inside the titanium tubes placed in the holes of said working electrode.

Preferably, the working electrode is treated according to known techniques, intended to improve the surface properties thereof. Thus, the internal surface 17 of the tubes may be treated to remove foreign materials therefrom, using suitable solvents, such as chlorinated hydrocarbons (e.g., methylene chloride, chloroform), ketones (e.g., methyl ethyl ketone, acetone) or chlorofluorocarbons. The above-described degreasing operation is followed by a mild acid micro-etching, using nitric acid at a concentration of about 5-10% v/v.

As shown in FIG. 3, the working electrode 11 is symmetrically positioned in the space between two counter electrodes 15, 15′ that are placed parallel to each other, the distance between said two counter electrodes being preferably in the range of 5 to 8 cm, and more preferably about 6 to 7 cm. Each of the counter electrodes 15, 15′ is preferably provided in the form of a plate, or a grid, the area of which being larger than the area of the perforated plate constituting the working electrode, thus avoiding electrical field edge effects which may lead to a deterioration of the plating properties of the titanium tubes mounted close to the edges of the working electrode. Preferably, the counter electrodes are made of a material selected from the group consisting of platinum, Platinized Pt and graphite. The counter electrodes and the working electrode are electrically connected (18, 18′) to the positive and negative poles of a power source, respectively (not shown).

Preferably, in order to assure sufficient concentration of samarium ions in the interior of the tubes placed in the holes of the working electrode, said working electrode is caused to oscillate backwards and forwards to and from each of said counter electrodes in turn. This oscillatory motion of the working electrode brings fresh portions of the solution into contact with the interior of the tubes. Typically, the rate of said oscillatory motion is about 5 to 20 cycles per minute. The oscillatory motion of the working electrode is combined with other modes of mixing of the solution, using, for example, suitable circulation means, which are preferably eductors for pumping and stirring, and filtration means.

According to a particularly preferred variant of the second embodiment, a technique known in the art as “Reverse Pulse Plating” (RPP) is advantageously applied, to improve the uniformity of the samarium deposit obtained inside the tubes. The technique is described in CircuiTree, Vol. 14(8), p. 28 (2001) and CircuiTree, Vol. 14(4), p. 52 (2001), which are incorporated herein entirely by reference. Thus, in a preferred embodiment, the method according to the invention comprises the steps of:
passing an electrical current of magnitude I_{forward} for a period of time t_{forward}, to electrochemically deposit elemental samarium inside the tubes;
reversing the polarity of the electrodes and passing a reverse current of magnitude Iᵣₑᵥₑᵣₛₑ for a period of time tᵣₑᵥₑᵣₛₑ, wherein I_{forward}<Iᵣₑᵥₑᵣₛₑ and t_{forward}>tᵣₑᵥₑᵣₛₑ,
reversing the polarity of the electrodes,
and repeating said steps to obtain a uniform deposit of samarium inside the tubes.

Preferably, I_{forward} has a current density in the range of 0.5 to 30 mA/cm², and preferably, in the range of 5 to 20 mA/cm². Preferably, the ratio Iᵣₑᵥₑᵣₛₑ:I_{forward} is in the range of 2:1 to 10:1, and preferably about 3:1.

Preferably, t_{forward} is in the range of 10 to 100 msec, and preferably about 40 msec, and tᵣₑᵥₑᵣₛₑ is in the range of 1 to 5 msec, and preferably about 2 to 3 msec.

At the end of the electrodeposition process, the samarium-containing titanium tubes are removed from the working electrode. Following sealing and activation as described above, they are ready for use as brachytherapy seeds.

While specific embodiments of the invention have been described for the purpose of illustration, it will be understood that the invention may be carried out in practice by skilled persons with many modifications, variations and adaptations, without departing from its spirit or exceeding the scope of the claims.

1. A method for densely packaging an element inside a casing, such that the element-containing casing is suitable for use in the preparation of a radiation source, comprising providing a solution containing ions of said element, positioning a working electrode and at least one counter electrode in contact with said solution, connecting said working electrode and said at least one counter electrode to the negative and positive poles of a power source, respectively, passing an electrical current between said electrodes to electrochemically deposit said element on said working electrode in a geometrical form corresponding to the form of the interior of the casing and concurrently or sequentially loading said casing with said element.

2. A method according to claim 1, wherein the casing is provided in the form of a cylindrically shaped canister, suitable for use in the preparation of an implantable radiation source for brachytherapy, and the element is electrochemically deposited in an essentially cylindrical form corresponding to the interior of said canister.

3. A method according to claim 2, wherein the element is a metal selected from the group consisting of samarium, thulium, palladium, tellurium and silver.

4. A method according to claim 4, wherein the metal is samarium.

5. A method according to claim 1, wherein the counter electrode positioned in the solution is in the form of a cylindrical surface, within which the working electrode, provided in the form of a wire, is coaxially arranged, whereby, following the passage of an electrical current between said electrodes, the element is electrochemically deposited on said wire, such that a solid body made of said element is obtained, said body having an essentially cylindrical form, wherein the symmetry axis of said body coincides with said wire.

6. A method according to claim 1, comprising positioning in the solution a working electrode provided in the form of a perforated plate, wherein each hole of said plate contains a casing made of a conductive material, the length and the cross-section of said casing being essentially the same as the thickness of said plate and cross-section of said hole, respectively, such that said casings are fixedly positioned in said holes, whereby, following the passage of an electrical current between said electrodes, the element is electrochemically deposited inside said casings.

7. A method according to claim 6, wherein the working electrode is symmetrically positioned in the space between two counter electrodes that are placed parallel to each other, and wherein said working electrode is caused to oscillate backwards and forwards towards and away from each of said counter electrodes in turn.

8. A method according to claim 6, wherein the passage of the electrical current is performed as follows:
passing an electrical current of magnitude I_{forward} for a period of time t_{forward}, to electrochemically deposit the element in the interior of the casings;
reversing the polarity of the electrodes and passing a reverse current of magnitude Iᵣₑᵥₑᵣₛₑ for a period of time tᵣₑᵥₑᵣₛₑ, wherein I_{forward}<Iᵣₑᵥₑᵣₛₑ and t_{forward}>tᵣₑᵥₑᵣₛₑ,
reversing the polarity of the electrodes,
and repeating said steps to obtain a uniform deposit of the element inside the casing.

9. A method for preparing a radiation source, comprising packaging an element inside a casing, such that the element-containing casing is suitable for use in the preparation of a radiation source according to claim 1, and further comprising the steps of sealing the casing and activating the same to convert the element contained in said casing into a radioactive isotope.

10. A samarium-containing canister produced by the method according to claim 4, for use in the preparation of an implantable radiation source for the treatment of tumors.

11. A radiation source comprising ¹⁴⁵Sm packed inside a casing, wherein said ¹⁴⁵Sm is obtained by neutron-irradiating ¹⁴⁴Sm, which was electrochemically deposited from a solution of a samarium salt, and wherein the density of said ¹⁴⁵Sm in said casing is greater than 1.5 g Sm/cm³.

12. A radiation source according to claim 11, wherein the density of ¹⁴⁵Sm in the casing is greater than 3 g Sm/cm³.

13. A radiation source according to claim 12, wherein the density of the samarium in the casing is greater than 5 g Sm/cm³.
